(19) United States
(12) Patent Application Publication
(10) Pub. No.: US 2020/0273555 A1
(43) Pub. Date: Aug. 27, 2020

(54) INTELLIGENT REMINDING METHOD, DEVICE AND ELECTRONIC APPARATUS

(71) Applicant: BOE TECHNOLOGY GROUP CO., LTD., Beijing (CN)

(72) Inventor: Zhenzhong ZHANG, Beijing (CN)

(21) Appl. No.: 16/713,818

(22) Filed: Dec. 13, 2019

(30) Foreign Application Priority Data
Feb. 25, 2019 (CN) 201910137871.0

(51) Int. Cl. G16H 20/10; G06N 5/04; G06N 20/00; A61J 7/04; G06F 40/279; G06F 40/30

(52) U.S. Cl. CPC G16H 20/10; G06N 5/04; G06F 40/30; A61J 7/0418; G06F 40/279; G06N 20/00

(57) ABSTRACT

An intelligent reminding method, an intelligent reminding device and an electronic apparatus are provided in the present disclosure. The method includes: acquiring first information input by a user; determining a user-intention template matching the first information, wherein the user-intention template comprises a plurality of fields; and judging whether the first information include contents corresponding to all the fields of the user-intention template.

### CROSS-REFERENCE TO RELATED APPLICATION

This application claims a priority to Chinese Patent Application No. 201910137871.0 filed in China on Feb. 25, 2019, the disclosure of which is incorporated herein in its entirety by reference.

### TECHNICAL FIELD

The present disclosure relates to the field of artificial intelligence technology, in particular to an intelligent reminding method, an intelligent reminding device and an electronic apparatus.

### BACKGROUND

With development of society and economy, improvement of science and technology, and improvement of people's living standard, a life expectancy of population is continuously increasing, and a population of old people in a total social population is increasing. Due to improvement of personal health and a quality of life of the old people, requirements of levels of personal care and health services are also increasing. For example, taking medicine on time or regular arrangements for daily life are usually of higher importance to the old people.

However, the old people often forget about health care issues due to memory loss or busy affairs, so an intelligent reminding method is needed. The intelligent reminding method may remind a user of relevant issues in time by interacting with the user, thereby meeting user needs.

In addition, for users who are busy with daily affairs and needing auxiliary tools to provide issues reminding services, there is also a demand for an intelligent reminding product.

### SUMMARY

In one aspect of the present disclosure, an intelligent reminding method is provided, including the following steps:

acquiring first information input by a user;

determining a user-intention template matching the first information, wherein the user-intention template includes a plurality of fields; and

judging whether the first information includes contents corresponding to all the fields of the user-intention template.

In some embodiments, the determining the user-intention template matching the first information includes:

acquiring text information corresponding to the first information;

classifying the text information; and

selecting the user-intention template matching the first information according to a classification result.

According to some embodiments of the present disclosure, the determining the user-intention template matching the first information includes:

acquiring text information corresponding to the first information;

extracting keywords of the text information corresponding to the first information; and

analyzing the extracted keywords, and selecting the user-intention template matching the first information according to an analysis result.

According to some embodiments of the present disclosure, the judging whether the first information includes the contents corresponding to all the fields of the user-intention template includes:

acquiring text information corresponding to the first information;

labeling the text information by using a preset algorithm to extract a content of at least one field corresponding to the user-intention template from the text information; and

judging whether the extracted content covers all the fields of the user-intention template.

According to some embodiments of the present disclosure, the user-intention template is a preset semantic template,

the judging whether the first information includes the contents corresponding to all the fields of the user-intention template includes:

extracting a content of at least one field from the first information according to the preset semantic template; and

judging whether the extracted content covers all the fields of the user-intention template.

According to some embodiments of the present disclosure, subsequent to the judging whether the first information includes the contents corresponding to all the fields of the user-intention template, the method further includes:

in a case that the first information includes the contents corresponding to all the fields of the user-intention template, setting a reminder message according to the first information;

or,

in a case that the first information does not include contents corresponding to all the fields of the user-intention template, determining a field to be supplemented;

sending the fields to be supplemented to the user;

receiving second information input by the user, and judging whether the second information includes a content of the field to be supplemented; and

when the second information includes the content of the field to be supplemented, setting a reminder message according to the first information and the second information.

In some embodiments, the judging whether the second information includes the content of the field to be supplemented includes:

performing matching and extraction on the second information and judging whether the second information includes the content corresponding to the field to be supplemented.

In some embodiments, the method further includes:

acquiring history log information of the user;

according to a content of the history log information and a content of the first information, determining whether to generate a conflict reminder message.

In some embodiments, the user-intention template is a medicine reminder template, and the history log information includes: medicine record information and health record information,

the determining whether to generate the conflict reminder message according to the content of the history log information and the content of the first information includes:

extracting key words of the medicine record information and the health record information; and

comparing the key words with the content of the first information, and determining whether to generate a medicine conflict reminder message.

In some embodiments, the first information is voice information or image information.

In another aspect of the present disclosure, a intelligent reminding device is provided, including:

a first acquiring module, configured to acquire first information input by a user;

a first determining module, configured to determine a user-intention template matching the first information, wherein the user-intention template includes a plurality of fields; and

a first judgment module, configured to judge whether the first information includes contents corresponding to all the fields of the user-intention template.

In some embodiments, the device further includes:

a setting module, configured to set a reminder message according to the first information in a case that the first information includes the contents corresponding to all the fields of the user-intention template.

In some embodiments, the device further includes:

a processing module, configured to determine a field to be supplemented in a case that the first information missing a content of at least one field of the user-intention template;

a sending module, configured to send the field to be supplemented to the user;

a second judgment module, configured to judge whether second information includes a content of the field to be supplemented;

wherein the sending module sets the reminder message according to the first information and the second information in a case that the second information including the content of the field to be supplemented;

wherein the first acquiring module is further configured to receive the second information input by the user.

In some embodiments, the device further includes:

a second acquiring module, configured to acquire history log information of the user;

a second determining module, configured to determine whether to generate a conflict reminder message according to a content of the history log information and a content of the first information.

In still another aspect of the present disclosure, an electronic apparatus is provided, including a processor and a memory, the memory is configured to store computer instructions, and the processor is configured to execute the computer instructions to perform the method for intelligent reminding according to any one of the foregoing embodiments.

In some embodiments, the first information is voice information, the electronic apparatus further includes a sound receiving device configured to receive the first information input by the user, wherein the processor is configured to convert the received voice information into corresponding text information.

In some embodiments, the electronic apparatus further comprising:

an information presenting device configured to present a reminder message under the control of the processor.

In some embodiments, the memory is configured to store a preset semantic template, and the processor is configured to extract a content of the first information according to the preset semantic template.

In some embodiments, the memory is configured to store a classifier formed by training based on a machine learning method, and the processor is configured to load the classifier to classify text information corresponding to the first information.

In yet still another aspect of the present disclosure, a computer readable storage medium is provided, on which is stored a computer program to be executed by a processor to perform the method for intelligent reminding according to any one of the foregoing embodiments.

### BRIEF DESCRIPTION OF THE DRAWINGS

FIG. 1 is a flow chart illustrating a method for intelligent reminding according to the embodiment of the present disclosure;

FIG. 2 is a flow chart illustrating another method for intelligent reminding according to the embodiment of the present disclosure;

FIG. 3 is a flow chart illustrating another method for intelligent reminding according to the embodiment of the present disclosure;

FIG. 4 is a flow chart illustrating another method for intelligent reminding according to the embodiment of the present disclosure;

FIG. 5 is a schematic structural diagram of a device for intelligent reminding according to the embodiments of the present disclosure;

FIG. 6 is a schematic structural diagram of another device for intelligent reminding according to the embodiments of the present disclosure;

FIG. 7 is a schematic structural diagram of another device for intelligent reminding according to the embodiments of the present disclosure; and

FIG. 8 illustrates a block diagram of an exemplary electronic apparatus suitable for implementing the embodiments of the present disclosure.

### DETAILED DESCRIPTION

The embodiments of the present disclosure will be described in detail hereinafter. Examples of the embodiments are shown in the drawings, same or similar reference numerals indicate same or similar elements or elements having same or similar functions. The embodiments described with reference to the drawings are exemplary and are intended to explain the present disclosure and should not be construed as limitations on the present disclosure.

An intelligent reminding method, an intelligent reminding device, and an electronic apparatus according to the embodiments of the present disclosure are described below with reference to the drawings.

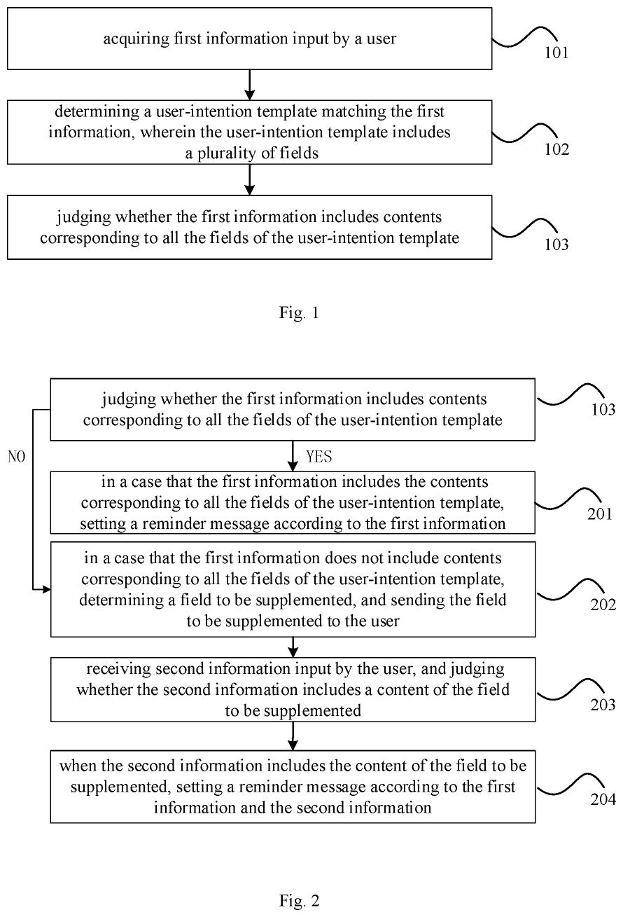

FIG. 1 is a flow chart illustrating a method for intelligent reminding according to the embodiment of the present disclosure, as shown in FIG. 1, the method includes the following steps.

Step 101, acquiring first information input by a user.

For example, the first information may be at least one of text information, voice information, and image information.

In some embodiments, the first information is the text information, and is input to an electronic apparatus described in the following embodiments of the present disclosure through an input method, for example.

In some embodiments, the first information is the voice information, which is input to the electronic apparatus described in the following embodiments of the present disclosure through a sound receiving device (such as a microphone, a pickup, etc.), and is converted into text by using a technology of voice-to-text.

In some embodiments, the first information may be an image, for example, a content written by the user on a paper, which is input to the electronic apparatus described in the following embodiments of the present disclosure through an imaging device (such as a camera, a scanner, etc.), and is converted to text by using an Optical character recognition (OCR).

A manner for the user inputting the first information is not particularly limited. For example, the user may directly operate the electronic apparatus to input information, or push information to the electronic apparatus via short message service, email, etc., or transfer information from a cloud to the electronic apparatus. Any applicable manner is available for inputting information.

Taking the voice information as an example, the first information may be input according to actual application needs of the user, for example, the user inputs a voice message through a microphone such as “Remember to remind me to take medicine on time tomorrow!” or “remember to remind me to get up tomorrow”.

It should be appreciated that, the user may start to input the voice information manually after manually triggering relevant keys, or may start to input the voice information after waking up a voice device through voice. The user may select according to an actual application, which is not limited in the solutions of the present disclosure.

Step 102, determining a user-intention template matching the first information, wherein the user-intention template includes a plurality of fields.

After acquiring the first information, a user intention incorporated in the first information may be analyzed in various manners, for example, by selecting an appropriate user-intention template. A preset user-intention template may match different types of information, and there is at least one field preset in the template, which may be used for targeted extraction of a content of the first information.

According to an example, text information corresponding to the first information is classified by a preset classifier, and the user-intention template matching the first information is selected according to a classification result.

According to another example, the text information corresponding to the first information is extracted by using a preset keyword extraction model, and keywords extracted are analyzed, and the user-intention template matching the first information is selected according to an analysis results.

Step 103, judging whether the first information includes contents corresponding to all the fields of the user-intention template.

In some embodiments, the above user-intention templates may be preset semantic templates corresponding to various user intentions. Different user intentions correspond to different semantic templates, such as a user intention A corresponds to a semantic template B, a user intention C corresponds to a semantic template D. The solutions of the present disclosure will be described below with an example of a semantic template set including at least a user-intention template “remind me to take medicine” and a user-intention template “remind me to get up”.

It should be also appreciated that, a semantic template includes all fields for realizing the user intention. For example, all the fields corresponding to the user-intention template “remind me to take medicine” include “time” and “medicine name”; for another example, all the fields corresponding to the user-intention template “remind me to get up” only include “time”.

According to each field determined and included in the user-intention template, the content extracted from the first information input by the user may be compared with these fields respectively, so as to judge whether the first information includes the contents of all the fields corresponding to the selected user-intention template.

Further, the judging whether the first information includes the contents corresponding to all the fields of the user-intention template, as a possible implementation, may be labelling the text information by using a preset algorithm, and extracting the content from the text information; and then judging whether the extracted content covers all the fields of the user-intention template elected; as another possible implementation, may be performing matching and extraction on the first information according to a preset semantic template (also referred to as a language model) (for example, performing matching and extraction on the voice information based on the language model); and judging whether the extracted content covers all the fields of the semantic template.

The above-mentioned “text information corresponding to the first information” may be text information input by the user, or text information converted through any appropriate manner, for example, text information obtained by converting voice information received through the technology of voice-to-text, or text information obtained by converting image information received through an OCR technology. An initial form and/or a conversion manner of the first information related in the present disclosure is not limited by the above examples.

Subsequent to step 103, when it is determined that the first information includes the contents of all the fields, a reminder message is set according to the content of the first information.

After determining that the first information includes the contents corresponding to all the fields, the reminder message is set according to the content of the first information. For example, the first information is voice information “remind me to eat aspirin at 8:00 am tomorrow”, and text information “remind me to eat aspirin at 8:00 am tomorrow” corresponding to the first information is labelled by using the preset algorithm, contents of each field extracted from the text information “remind me to eat aspirin at 8:00am tomorrow” are “8:00 am tomorrow ” and “aspirin” respectively; since the extracted contents include all the contents corresponding to all the fields “time” and “medicine name”, the reminder message may be set according to “8:00 am tomorrow ” and “aspirin”.

The intelligent reminding method based on semantic analysis in the embodiments of the present disclosure determines the user-intention template matching the first information by acquiring the first information input by the user, wherein the user-intention template includes the plurality of fields; and judges whether the first information includes the contents corresponding to all the fields of the user-intention template. When it is determined that the first information includes the contents of all the fields, the reminder message is set according to the content of the first information. Thus, by acquiring the first information input by the user, the user-intention template corresponding to the first information input is determined, various fields information corresponding to user's specific needs are analyzed and extracted, and the reminder message is set accordingly to meet the user's needs, thereby improving a using experience.

Based on the above embodiments, it should be also appreciated that, if the first information does not include the contents of all the fields, the user needs to be further reminded to continue to input related information to acquire the contents corresponding to all the fields of the user-intention template, so as to complete the setting of the reminder message, which is described in detail according to one specific example of the present disclosure with reference to FIG. 2.

FIG. 2 is a flow chart illustrating a method for intelligent reminding according to the embodiment of the present disclosure, as shown in FIG. 2, subsequent to step 103, the method further includes:

step 201, in a case that the first information includes the contents corresponding to all the fields of the user-intention template, setting a reminder message according to the first information; or

step 202, in a case that the first information does not include contents corresponding to all the fields of the user-intention template, determining a field to be supplemented, and sending the field to be supplemented to the user.

Optionally, when it is determined that the first information does not include the contents corresponding to all the fields, the field to be supplemented is determined. For example, the text information corresponding to the first information is “remind me to take medicine at 8:00 am tomorrow”, and the text information “remind me to take medicine at 8:00 am tomorrow” is labelled by the preset algorithm. Since a content “8:00 am tomorrow” extracted from the text information “remind me to take medicine at 8:00 am tomorrow” only includes a “time” field, so the field to be supplemented is “medicine name”.

After determining the field to be supplemented, the corresponding a reminder message (such as a voice or a text) may be generated according to the field to be supplemented to remind the user, or the field to be supplemented may be notified to the user by displaying reminder input information on a corresponding display screen. Continue to take the above example as an example, as a possible implementation, the user is reminded to continue to input “what medicine to take” through voice interaction, that is, request the user to provide information about “medicine name”.

Step 203, receiving second information input by the user, and judging whether the second information includes a content of the field to be supplemented.

Step 204, when the second information includes the content of the field to be supplemented, setting a reminder message according to the first information and the second information.

For example, the receiving the second information input by the user, and judging whether the second information includes the content of the field to be supplemented, as a possible implementation, may be labelling text information corresponding to the second information by using the preset algorithm, and extracting a corresponding content from the labelled text information; and then judging whether the extracted content belongs to the field to be supplemented; as another possible implementation, may be performing matching and extraction on the second information according to the preset language model (for example, perform matching and extraction on a content of the voice information based on the language model); and judging whether extracted content belongs to the field to be supplemented.

Therefore, when it is determined that the second information includes the content corresponding to the field to be supplemented, the reminder message is set according to all the contents received successively. In the above example, the corresponding a reminder message is set according to the contents extracted from the first information and the second information. For example, the second information is voice information “aspirin”, and matching and extraction are performed on the voice information “aspirin” through the preset language model, and it is determined that the extracted content includes the corresponding information “aspirin” of the field to be supplemented “medicine name”, then the reminder message is set according to “8:00 am tomorrow ” and “aspirin”.

It should be appreciated that, when judging that the second information does not include the content corresponding to the field to be supplemented, the user may be continued to be notified of a need for supplementing information until the contents of all the fields required for setting the reminder message can be acquired from information input by the user.

Thus, the users' needs may be acquired through a plurality of voice interactions or any other appropriate interactions with the user, and relevant setting reminders may be used for meeting the users' needs, so as to accurately grasp the users' needs and improve the using experience.

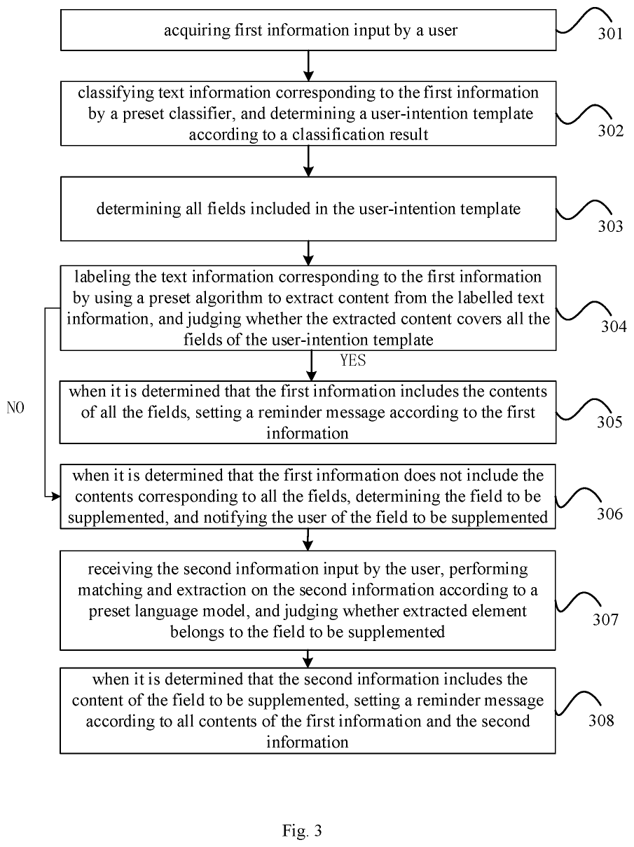

For a person skilled in the art to be more aware of an implementation process of the solutions of the present disclosure, the intelligent reminding method of the embodiments of the present disclosure will be described in detail below with reference to FIG. 3.

FIG. 3 is a flow chart illustrating a method for intelligent reminding according to the embodiment of the present disclosure, as shown in FIG. 3, the method includes the following steps.

Step 301, acquiring first information input by a user.

Step 302, classifying text information corresponding to the first information by a preset classifier, and determining a user-intention template according to a classification result.

Step 303, determining all fields included in the user-intention template.

Step 304, labeling the text information corresponding to the first information by using a preset algorithm to extract a content from the labelled text information, and judging whether the extracted content covers all the fields of the user-intention template.

Step 305, when it is determined that the first information includes the contents of all the fields, setting a reminder message according to the first information.

According to some embodiments of the present disclosure, various semantic templates corresponding to different user intentions may be defined in advance; input information may be acquired through an interactive dialogue between an electronic apparatus and the user and analyzed to acquire the semantic templates corresponding to the user intentions.

For example, two types of user-intention templates are defined in advance, which are “remind me to take medicine” and “remind me to get up”, and the corresponding semantic templates are {“time”, “drug name”} and {“wake up time”}. These semantic templates are respectively includes all the fields needed to realize the user intentions of “remind me to take medicine” and “remind me to get up”. A first judgment module fills values of each field in the semantic template by analyzing the first information input by the user. For example, the first information input by the user is “wake me up at 8:00 am tomorrow”, a user-intention template “remind me to get up” is determined firstly, and then a “wake up time” of the semantic template is filled to realize the user intention according to a corresponding semantic template, a time “8:00 am tomorrow” is acquired from the first information “wake me up at 8:00 am tomorrow”, at this time, the semantic template is completed.

User intention analysis may be achieved through a classifier formed by a machine learning training, that is, labeling a certain amount of training corpora (a training sample includes a large amount of correspondences between information and the user intentions), using the corpora to train the classifier (such as a Support Vector Machine (SVM) classifier or a classifier based on neural network), and then using the classifier to judge the user intention. For example, supposing a trained classifier is C. When determining the first information “wake me up at 8:00 am tomorrow”, the classifier is used for classifying: C (“wake me up at 8:00 am tomorrow”)=“remind me to get up”, a type is a type of the user intention predefined.

For example, the training corpora may further include different expressions of multiple users for a same user intention in life, for example, for the user intention “remind me to take medicine”, the different expressions may be “remind me to take medicine tomorrow”, “I need to take medicine tomorrow” , “Do I need to take medicine tomorrow?”, etc., that is, a completeness of the classifier is ensured by acquiring a large number of training corpora corresponding to different user intentions for training, so as to better meet the user needs.

After analyzing and determining the user-intention template, a corresponding content is extracted from the first information input by the user and filled into the semantic template corresponding to the user intention. Specifically, the text information corresponding to the first information is labelled in sequence by using the preset algorithm, such as a Conditional Random Field (CRF), from which corresponding subsequences are extracted. For example, for the above example, the field to be supplemented is “wake up time”, the text information “wake me up at 8:00 am tomorrow ” is labelled in sequence by using the CRF to acquire a plurality of subsequences with the contents of “wake”, “me”, “up” and “8:00 am tomorrow” in sequence, a time “8:00 am tomorrow” is extracted, and then a specific date of tomorrow morning is determined according to a system time (for example, today is Jan. 1, 2018 and tomorrow is Jan. 2, 2018),which is filled into the template.

It should be appreciated that, subsequent to step 304, step 305 or steps 306 to 308 may be performed to determine whether the first information includes the contents corresponding to all the fields.

Step 306, when it is determined that the first information does not include the contents corresponding to all the fields, determining the field to be supplemented, and notifying the user of the field to be supplemented.

Step 307, receiving the second information input by the user, performing matching and extraction on the second information according to a preset language model, and judging whether extracted element belongs to the field to be supplemented.

Step 308, when it is determined that the second information includes the content of the field to be supplemented, setting a reminder message according to all contents of the first information and the second information.

That is to say, in the above example, if a corresponding content sequence cannot be extracted from the first information currently input by the user, a question for the user is generated. For example, when the user says “remind me to take medicine at 8:00 am tomorrow”, the user intention template is determined to be “remind me to take medicine”, and a template to be filled is {“time”, “medicine name”}. From the first information input by the user, a content of the time “8:00 am tomorrow” may be extracted, but the “medicine name” may not be known. At this time, a question for the user “what is the medicine name please?” is generated, if “aspirin” is input by the user, the medicine name is extracted and filled, and a cycle of generating the question for the user and inputting an answer by the user is continued until the semantic template is filled.

Information input by the user again is usually a purposeful input for the field to be supplemented. The language model may be trained according to the corpora after multiple interactions, so as to improve an efficiency of performing the matching and the extraction on the second information, which is convenient for the user.

According to the embodiments of the present disclosure, the intelligent reminding device may interact with the user in a natural language manner, analyze the intention expressed by the user, and then help the user complete required task, meet the user's needs, and improve the using experience.

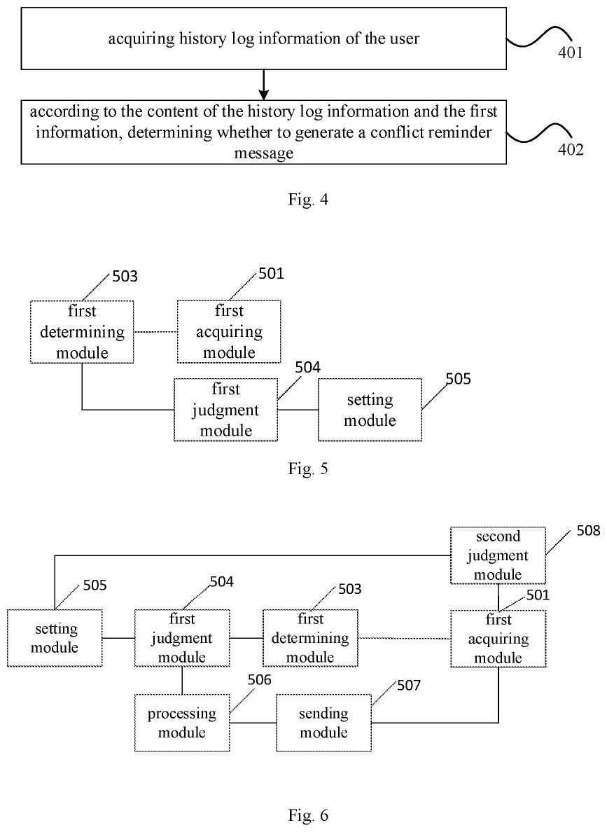

The following specifically describes with reference to FIG. 4 that the intelligent reminding method based on semantic analysis in this embodiment may help users, especially elderly users, improve an efficiency of health management.

FIG. 4 is a flow chart illustrating a method for intelligent reminding according to the embodiment of the present disclosure, as shown in FIG. 4, the method includes the following steps.

Step 401, acquiring history log information of the user.

Step 402, according to a content of the history log information and a content of the first information, determining whether to generate a conflict reminder message.

The historical log information of the user includes one or more of the following information, such as operation record information, health record information, and medicine record information, which may be extracted according to actual application needs.

Specifically, there are many conditions where determining whether to generate the conflict reminder message according to the content of the historical log information and the content of the first information input by the user, examples are as follows.

As a possible implementation, the user intention is to remind the user to get up, the history log information includes: a user's historical setting time is 8:00 am to remind the user to get up, and a content of information input by the user is 7:00 am tomorrow, which conflicts with a user's operation habits and the conflict reminder message may be generated to remind the user again to confirm whether to change or not.

As another possible implementation, the user intention is to remind the user to take medicine, the history log information includes the health record information and the medicine record information, and the determining whether to generate the conflict reminder message according to the content of the historical log information and the content of the first information input by the user includes: extracting key words of the medicine record information and the health record information; and comparing the key words with the content of the first information, and determining whether to generate a medicine conflict reminder message.

A health knowledge base may be set in advance to record and store relevant medical knowledge and user health record information. For example, store interactions between medicines or medicine ingredients, record health information of the user (for example, a treatment being taken and a medicine being taken by the user, etc.), so as to view the medicine being taken by the user and a corresponding allergy history by searching the health knowledge base, and determine whether a side effect occurs between a medicine to be taken and the medicine being taken or whether the user is allergic to the medicine to be taken. If the side effect occurs between the medicine to be taken and the medicine being taken or the user is allergic to the medicine to be taken, an alert to the user is generated; if the side effect does not occur between the medicine to be taken and the medicine being taken or the user is not allergic to the medicine to be taken, the reminder message is set.

It should be appreciated that some users, such as old people, often forget about health care issues due to memory loss or busy affairs. The intelligent reminding method based on semantic analysis provided in the present disclosure may remind the users to complete relevant issues on time. Since many old people suffer from various diseases, and need to take many medicines at the same time, many old people are not aware of conflicts between medicine ingredients. The intelligent reminding method based on semantic analysis provided in the present disclosure may remind the users of potential conflict risks of the medicine ingredients. At the same time, a natural language interaction manner adopted by the present disclosure is more convenient to operate than a conventional manner of setting an alarm manually.

In order to achieve the above purpose, a device for intelligent reminding is further provided in the present disclosure.

FIG. 5 is a schematic structural diagram of a device for intelligent reminding according to the embodiments of the present disclosure, as shown in FIG. 5, the device includes a first acquiring module 501, a first determining module 503, a first judgment module 504 and a setting module 505.

The first acquiring module 501 is configured to acquire first information input by a user;

The first determining module 503 is configured to determine a user-intention template matching the first information, wherein the user-intention template includes a plurality of fields.

The first judgment module 504 is configured to judge whether the first information includes contents corresponding to all the fields of the user-intention template.

The setting module 505 is configured to set a reminder message according to the first information in a case that the first information includes the contents corresponding to all the fields of the user-intention template.

According to some embodiments, the first determining module 503 is configured to classify text information corresponding to the first information; and select the user-intention template matching the first information according to a classification result.

The device shown in FIG. 6 further includes a processing module 506, a sending module 507 and a second judgment module 508.

The processing module 506 is configured to determine a field to be supplemented in a case that the first information missing a content of at least one field of the user-intention template.

The sending module 507 is configured to send the field to be supplemented to the user.

The second judgment module 508 is configured to judge whether second information includes a content of the field to be supplemented.

The setting module 505 is further configured to set a reminder message according to the first information and the second information when it is determined that the second information includes the content corresponding to the field to be supplemented.

Further, the first judgment module 504 is configured to label the text information corresponding to the first information; and judge whether the labelled text information includes the contents corresponding to all the fields of the user-intention template.

Further, the second judgment module 508 is configured to perform matching and extraction on the second information and judge whether a content extracted corresponds to the field to be supplemented.

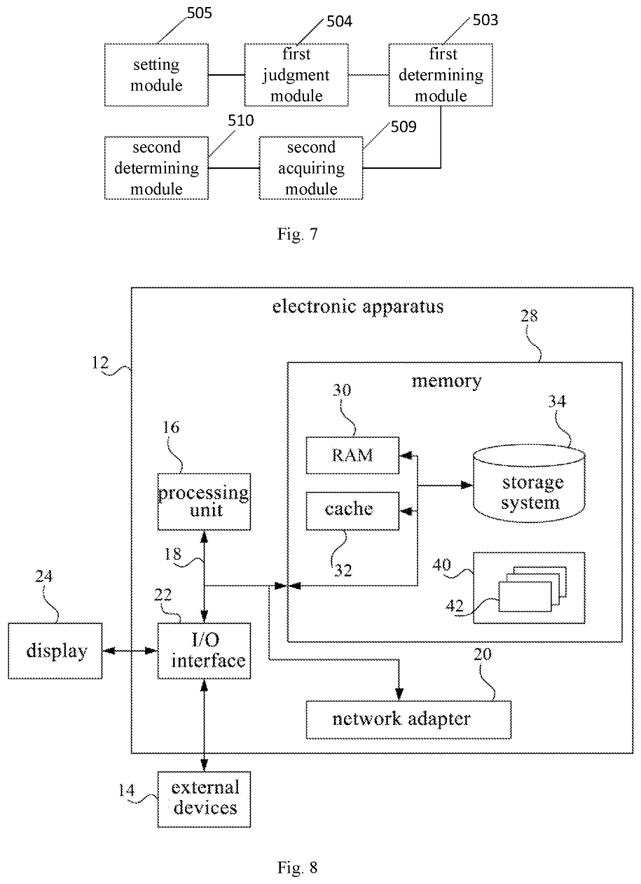

The device shown in FIG. 7 further includes a second acquiring module 509 and a second determining module 510.

The second acquiring module 509 is configured to acquire history log information of the user.

The second determining module 510 is configured to determine whether to generate a conflict reminder message according to a content of the history log information and a content of the first information.

Further, the user intention is to remind the user to take medicine, the history log information includes the health record information and the medicine record information, the second determining module 510 is configured to extract key words of the medicine record information and the health record information; compare the key words with the content of the first information, and determine whether to generate a medicine conflict reminder message.

It should be appreciated that explanations about the intelligent reminding method based on semantic analysis in the foregoing embodiments are also applicable to the intelligent reminding device based on semantic analysis in this embodiment, which are not repeated herein.

The modules in this embodiment are logic function modules, which are implemented by a processor having a data processing capability and/or a program execution capability executing computer instructions with corresponding functions. The processor includes, but is not limited to, one or more of a central processing unit (CPU), a micro controller unit (MCU), a digital signal processor (DSP), an application specific integrated circuit (ASIC)\field-programmable gate array (FPGA), and a tensor processing unit (TPU). Each module may be implemented by including one or more chips of the above devices.

The intelligent reminding device based on semantic analysis in the embodiments of the present disclosure determines the user-intention template matching the first information by acquiring the first information input by the user, wherein the user-intention template includes the plurality of fields; and judges whether the first information includes the contents corresponding to all the fields of the user-intention template. When it is determined that the first information includes the content of all the fields, the reminder message is set according to the first information. Thus, by acquiring the first information input by the user, the user-intention template corresponding to the first information input is determined, various fields information corresponding to user's specific needs are analyzed and extracted, and the reminder message is set accordingly to meet the user's needs, thereby improving the using experience.

In order to implement the above embodiments, an electronic apparatus is further provided in the present disclosure, including a processor and a memory, the processor is configured to execute a program corresponding to an executable program code by reading the executable program code stored in the memory, to perform the method for intelligent reminding according to any one of the foregoing embodiments.

In order to implement the above embodiments, a computer program product is further provided in the present disclosure, a processor is configured to execute instructions in the computer program product to perform the method for intelligent reminding according to any one of the foregoing embodiments.

In order to implement the above embodiments, a computer readable storage medium is provided, on which is stored a computer program to be executed by a processor to perform the method for intelligent reminding according to any one of the foregoing embodiments.

FIG. 8 illustrates a block diagram of an exemplary electronic apparatus suitable for implementing the embodiments of the present disclosure. An electronic apparatus 12 shown in FIG. 8 is only an example, and should not impose any limitation on functions and a scope of use of the embodiments of the present disclosure.

The electronic apparatus may be, for example, a mobile phone, a tablet computer, a personal computer, a personal digital assistant, and the like.

As shown in FIG. 8, the electronic apparatus 12 is expressed in a form of a general computing device. Components of the electronic apparatus 12 may include, but are not limited to, one or more processors 16, a memory 28, and a bus 18 connecting different system components (including the memory 28 and the processor 16).

The bus 18 represents one or more of several types of bus structures, including a memory bus or a memory controller, a peripheral bus, an accelerated graphics port, a processor, or a local area bus of any bus structure having a variety of bus structures. For example, these architectures include, but are not limited to, an Industry Standard Architecture (ISA) bus, a Micro Channel Architecture (MAC) bus, an enhanced ISA bus, a Video Electronics Standards Association (VESA) local area bus and a Peripheral Component Interconnection (PCI) bus.

The electronic apparatus 12 typically includes a variety of computer system readable media. These media may be any available media that may be accessed by the electronic apparatus 12, including volatile and non-volatile media, removable and non-removable media.

The Memory 28 may include a computer system readable medium in a form of volatile memory, such as a random access memory (RAM) 30 and/or a cache memory 32. The electronic apparatus 12 may further include other removable/non removable, volatile/non-volatile computer system storage media. For example, a storage system 34 may be configured to read and write a non-removable, non-volatile magnetic medium (not shown in FIG. 8, commonly referred to as “a hard drive”). Although not shown in FIG. 8, a disk drive for performing reading and writing on a removable non-volatile disk (such as “a floppy disk”) and an optical disk for performing reading and writing on a removable non-volatile disc (such as a compact disc read only memory (CD-ROM)) and a digital video disc read only memory (DVD-ROM) may be provided. In these cases, each drive may be connected to the bus 18 through one or more data medium interfaces. The memory 28 may include at least one program product having a set (e.g., at least one) of program modules configured to perform the functions of the embodiments of the present application.

A program/utility tool 40 having a set (at least one) of program modules 42 may be stored in, for example, the memory 28. Such program modules 42 include, but are not limited to, an operating system, one or more application programs, other program modules, and program data (e.g., the semantic templates, the classifiers, etc. are stored in the memory 28), each or some combination of these examples may include an implementation of a network environment. The program modules 42 generally perform the functions and/or the methods in the embodiments described in this application.

The electronic apparatus 12 may further communicate with one or more external devices 14 (such as a keyboard, a pointing device, a display 24, etc.), and may further communicate with one or more devices that enable the user to interact with a computer system/server 12, and/or communicate with any device (such as a network card, a modem, etc.) that enables the computer system/server 12 to communicate with one or more other computing devices. This communication may be performed through an input/output (I/O) interface 22. In addition, the electronic apparatus 12 may further communicate with one or more networks (such as a local area network (LAN), a wide area network (WAN), and/or a public network (such as an Internet) through a network adapter 20. As shown in FIG. 8, the network adapter 20 communicates with other modules of the electronic apparatus 12 through the bus 18. It should be appreciated that although not shown in FIG. 8, other hardware and/or software modules, including but not limited to: microcode, device drivers, redundant processing units, external disk drive arrays, RAID systems, tape drives and data backup storage systems, may be used in combination with the electronic apparatus 12.

Optionally, the external devices 14 of the electronic apparatus include a sound receiving device (such as a pickup or a microphone) configured to receive voice information expressed by the user, wherein the processor is configured to convert the received the voice information received into corresponding text information.

Optionally, the external devices 14 of the electronic apparatus include an information presenting device (such as a speaker, a display screen, etc.) configured to present a reminder message corresponding to a user intention under the control of the processor, for example, a reminder is performed in a form of audio, image, text, video, or the like, or the reminder is performed in an appropriate combination of multiple forms, which is not limited in the present disclosure.

The processor 16 executes various functional applications and data processing by running programs stored in the memory 28, for example, the processor 16 implements the methods mentioned in the foregoing embodiments.

In the description of the present disclosure, it should be appreciated that, terms “first” and “second” are only used for descriptive purposes, and cannot be understood as indicating or implying relative importance or implicitly indicating the number of technical features indicated. Therefore, features defined including “first” and “second” may explicitly or implicitly include at least one of the features. In the description of the present disclosure, a meaning of “a plurality of” is at least two, for example, two, three, etc., unless it is specifically defined otherwise.

In the description of the specification, descriptions of reference terms “one embodiment”, “some embodiments”, “examples”, “specific examples”, or “some examples” and the like mean specific features, structures, materials, or characteristics described in combination with the embodiments or examples are included in at least one embodiment or example of the present disclosure. In this specification, schematic expressions of the above terms are not necessarily directed to a same embodiment or example. Furthermore, the specific features, structures, materials, or characteristics described may be combined in any suitable manner in any one or more embodiments or examples. In addition, without any contradiction, a person skilled in the art may combine different embodiments or examples and features of the different embodiments or examples described in this specification.

Although the embodiments of the present disclosure have been shown and described above, it should be appreciated that, the above embodiments are exemplary and should not be construed as limitations on the present disclosure, a person skilled in the art may make various changes, modifications, substitution and variants over the above embodiments without departing from the spirit of the present disclosure.

What is claimed is:

1. A method for intelligent reminding, comprising:
acquiring first information input by a user;
determining a user-intention template matching the first information, wherein the user-intention template comprises a plurality of fields; and
judging whether the first information comprises contents corresponding to all the fields of the user-intention template.

2. The method according to claim 1, wherein the determining the user-intention template matching the first information comprises:
acquiring text information corresponding to the first information;
classifying the text information; and
selecting the user-intention template matching the first information according to a classification result.

3. The method according to claim 1, wherein the determining the user-intention template matching the first information comprises:
acquiring text information corresponding to the first information;
extracting keywords of the text information corresponding to the first information; and
analyzing the extracted keywords, and selecting the user-intention template matching the first information according to an analysis result.

4. The method according to claim 1, wherein the judging whether the first information comprises contents corresponding to all the fields of the user-intention template comprises:
acquiring text information corresponding to the first information;
labeling the text information by using a preset algorithm to extract a content corresponding to at least one field of the user-intention template from the text information; and
judging whether the extracted content covers all the fields of the user-intention template.

5. The method according to claim 1, wherein the user-intention template is a preset semantic template,
the judging whether the first information comprises the contents corresponding to all the fields of the user-intention template comprises:
extracting a content of at least one field from the first information according to the preset semantic template; and
judging whether the extracted content covers all the fields of the user-intention template.

6. The method according to claim 1, subsequent to judging whether the first information comprises the contents corresponding to all the fields of the user-intention template, further comprising:
in a case that the first information comprises the contents corresponding to all the fields of the user-intention template, setting a reminder message according to the first information;
or,
in a case that the first information does not comprise contents corresponding to all the fields of the user-intention template, determining a field to be supplemented;
sending the field to be supplemented to the user;
receiving second information input by the user, and judging whether the second information comprises a content of the field to be supplemented; and
when the second information comprises the content of the field to be supplemented, setting a reminder message according to the first information and the second information.

7. The method according to claim 6, wherein judging whether the second information comprises the content of the field to be supplemented comprises:
performing matching and extraction on the second information and judging whether the second information comprises the content corresponding to the field to be supplemented.

8. The method according to claim 1, further comprising:
acquiring history log information of the user;
according to a content of the history log information and the content of the first information, determining whether to generate a conflict reminder message.

9. The method according to claim 8, wherein the user-intention template is a medicine reminder template, and the history log information comprises: medicine record information and health record information,
the determining whether to generate the conflict reminder message according to the content of the history log information and the content of the first information comprises:
extracting key words of the medicine record information and the health record information;
comparing the key words with the content of the first information, and determining whether to generate a medicine conflict reminder message.

10. The method according to claim 1, wherein the first information is voice information or image information.

11. A device for intelligent reminding, comprising:
a first acquiring module, configured to acquire first information input by a user;
a first determining module, configured to determine a user-intention template matching the first information, wherein the user-intention template comprises a plurality of fields; and
a first judgment module, configured to judge whether the first information comprises contents corresponding to all the fields of the user-intention template.

12. The device according to claim 11, further comprising:
a setting module, configured to set a reminder message according to the first information in a case that the first information comprises the contents corresponding to all the fields of the user-intention template.

13. The device according to claim 12, the first acquiring module is further configured to receive a second information input by the user; the sending module further sets the reminder message according to the first information and the second information in a case that the second information comprising the content of the field to be supplemented;
the device further comprises:
a processing module, configured to determine a field to be supplemented in a case that the first information missing a content of at least one field of the user-intention template;
a sending module, configured to send the field to be supplemented to the user;
a second judgment module, configured to judge whether the second information comprises a content of the field to be supplemented.

14. The device according to claim 11, further comprising:
a second acquiring module, configured to acquire history log information of the user;
a second determining module, configured to determine whether to generate a conflict reminder message according to a content of the history log information and a content of the first information.

15. An electronic apparatus, comprising a processor and a memory, the memory is configured to store computer instructions, and the processor is configured to execute the computer instructions to perform the method according to claim 1.

16. The electronic apparatus according to claim 15, wherein the first information is voice information, the electronic apparatus further comprises a sound receiving device configured to receive the first information input by the user, wherein the processor is configured to convert the received voice information into corresponding text information.

17. The electronic apparatus according to claim 15, further comprising:
an information presenting device configured to present a reminder message under the control of the processor.

18. The electronic apparatus according to claim 15, wherein the memory is configured to store a preset semantic template, and the processor is configured to extract a content of the first information according to the preset semantic template.

19. The electronic apparatus according to claim 15, wherein the memory is configured to store a classifier formed by training based on a machine learning method, and the processor is configured to load the classifier to classify text information corresponding to the first information.

20. A computer readable medium on which is stored a computer program to be executed by a processor to perform the method according to claim 1.
